(19) United States
(12) Patent Application Publication
(10) Pub. No.: US 2013/0096510 A1
(43) Pub. Date: Apr. 18, 2013

(54) CODED FERRULE FOR A DRUG DELIVERY DEVICE

(71) Applicants: David Plumptre, Worcestershire (GB); Richard James Vincent Avery, Gloucestershire (GB); Joseph Butler, Warwickshire (GB)

(72) Inventors: David Plumptre, Worcestershire (GB); Richard James Vincent Avery, Gloucestershire (GB); Joseph Butler, Warwickshire (GB)

(73) Assignee: Sanofi-Aventis Deutschland GmbH, Frankfurt am Main (DE)

(21) Appl. No.: 13/641,804

(22) PCT Filed: Apr. 21, 2011
(86) PCT No.: PCT/EP2011/056479

§ 371 Date: Dec. 21, 2012

Related U.S. Application Data
(60) Provisional application No. 61/327,293, filed on Apr. 23, 2010.

(30) Foreign Application Priority Data
Jul. 29, 2010 (EP) 10171167.9

(51) Int. Cl. A61M 5/31

(52) U.S. Cl. CPC A61M 5/3129
USPC 604/189

(57) ABSTRACT

A ferrule and a cartridge for use with a drug delivery device. A neck portion of the cartridge comprises a first coding feature. The ferrule comprises a main body defining a bore that extends from a proximal end and a distal end of the main body. The bore is configured for receiving the cartridge. A pass through is defined by the distal end. A coding feature is provided along at least a portion of the main body of the ferrule. This coding feature cooperates with a corresponding coding feature provided by a drug delivery device. Further, the ferrule is configured to cooperate with the first coding feature of the neck portion of the cartridge.

### CROSS REFERENCE TO RELATED APPLICATIONS

The present application is a U.S. National Phase Application pursuant to 35 U.S.C. §371 of International Application No. PCT/EP2011/056479 filed Apr. 21, 2011, which claims priority to U.S. Provisional Patent Application No. 61/327,293 filed Apr. 23, 2010 and European Patent Application No. 10171167.9 filed Jul. 29, 2010. The entire disclosure contents of these applications are herewith incorporated by reference into the present application.

### FIELD OF INVENTION

Specific embodiments of this disclosure relate to reservoirs, particularly reservoirs containing a medicament. More particularly, the present application is generally directed to a coded ferrule for use with a reservoir and a reservoir holder so as to prevent unwanted reservoir cross use. As just one example, such medicament reservoirs may comprise an ampoule, a cartridge, a vial, or a pouch, and may be used with a medical delivery device. Exemplary medical delivery devices include, but are not limited to, syringes, pen-type injection syringes, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient.

The term “medicament”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound, wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

For example, with respect to insulin, a patient suffering from diabetes may require a certain amount of insulin to either be injected via a pen-type injection syringe or infused via a pump. With respect to certain known reusable pen-type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder may be disconnected from the drug delivery device and the empty cartridge may be removed and replaced with a new cartridge. Most suppliers of such cartridges recommend the user to dispose of the empty cartridges properly. Where the drug delivery device comprises a disposable device, once the cartridge is empty, the user may be recommended to dispose of the entire device.

Such known self administration systems requiring the removal and reloading of empty cartridges have certain limitations. For example, in certain generally known systems, a user simply loads a new cartridge into the delivery system without the drug delivery device or without the cartridge having any mechanism of preventing cross-use of an incorrect cartridge. That is, the drug delivery device does not have a mechanism for determining whether the medicament contained in the cartridge is indeed the correct type of medicament to be administered by the patient. Alternatively, certain known drug delivery devices do not present a mechanism for determining whether the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short-acting insulin in lieu of a long-acting insulin could result in injury or even death.

Some drug delivery devices or systems may use a color coding scheme to assist a user or care giver in selecting the correct cartridge to be used with a drug delivery device. However, such color coding schemes may pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and are manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge to its drug type and design an injection device that only accepts or works with the dedication or coded features provided on or with the cartridge so as to prevent unwanted cartridge cross use. Similarly, there is also a general need for a dedicated cartridge that may allow the medical delivery device to be used with only an authorized cartridge containing a specific medicament while also preventing undesired cartridge cross-use.

There is also a general need to provide a dedicated cartridge that is difficult to tamper with so that the cartridge may not be compromised in that the cartridge can be used with an unauthorized drug or drug delivery device. Because such cartridges may be difficult to tamper with, they may also reduce the risk of counterfeiting: i.e. making it more difficult for counterfeiters to provide unregulated counterfeit medicament carrying products.

One problem to be solved by the present disclosure is to provide a ferrule, a cartridge and a drug delivery device which contribute to increasing the safety of the user.

### SUMMARY

One aspect relates to a ferrule for use with a cartridge. The ferrule may comprise a main body. Said main body may have a length generally equivalent to a length of a tubular barrel of said cartridge. The main body may define a bore. The bore may extend between a proximal end and a distal end of the main body. The bore may be configured for receiving the cartridge. A side wall may extend from the proximal end and the distal end. The ferrule may comprise a pass through. The pass through may be defined by the distal end. The ferrule may comprise at least one, preferably two ore more, coding features. The at least one coding feature may be provided along at least a portion of the main body. The coding feature may cooperate with a corresponding coding feature provided by a drug delivery device. In particular, said coding feature may cooperate with a corresponding coding feature provided by a cartridge holder of said drug delivery device. Additionally or alternatively, said coding feature may cooperate with a corresponding coding feature provided by a dose setting member of said drug delivery device.

According to an embodiment, the ferrule comprises a molded ferrule. Alternatively, the ferrule may comprise a metallic ferrule. Said ferrule may comprise a heat shrink material.

According to an embodiment, said coding feature is provided along an end face of said main body. Alternatively, the coding feature may be provided along a side wall of said main body. In particular, said coding feature may extend from said proximal end to said distal end.

According to an embodiment, said coding feature comprises a plurality of indentations. The plurality of indentations may be provided on the end face of said main body. Alternatively, said coding feature comprises a plurality of protrusions. The plurality of protrusions may be provided on the end face of said main body.

According to an embodiment, said ferrule further comprises a, preferably, releasable coupling for mounting a needle assembly. Said releasable coupling may comprise a thread.

According to an embodiment, said coding feature comprises slots. The slots may be provided along said side wall of said main body. Said slots may extend from said proximal end to said distal end of said main body.

According to an embodiment, said coding feature comprises a vertical wall of said main body. Said vertical wall may comprise a varying thickness. Said vertical wall comprising said varying thickness may comprise at least one flat surface.

According to an embodiment, the ferrule may comprise a flange, preferably an inner flange. Said coding feature may be provided near the inner flange of said ferrule.

According to an embodiment, said corresponding coding feature of said drug delivery device is provided near a distal end of a cartridge holder of said drug delivery device.

According to an embodiment, said coding feature prevents said cartridge from rotating within said cartridge holder. Additionally or alternatively, said coding feature may prevent said ferrule from rotating with respect to said cartridge.

According to an embodiment, at least one protrusion may be provided in said bore of said main body. Said at least one protrusion may grip behind a neck of said cartridge when said ferrule is mounted onto said cartridge.

According to an embodiment, a membrane may be provided. Said membrane may be located between said pass through defined by said distal end of said main body and said cartridge.

A further aspect relates to, a ferrule for use with a cartridge. Said ferrule may comprise a main body. The main body may define a bore. Said main body may extend from a proximal end to a distal end. Said bore may be configured for receiving said cartridge. A proximal flange may be located near said proximal end of said main body. A side wall may extend from said flange towards said distal end. A pass through may be defined by said distal end. A coding feature may be provided along said distal end of said main body of said ferrule. Said coding feature may cooperate with a corresponding coding feature provided by a drug delivery device.

According to an embodiment, said coding feature comprises a beveled edge. The beveled edge may be located near said distal end of said main body.

In another arrangement, a cartridge for use with a drug delivery device comprises a tubular member or barrel. Said tubular member may comprise a glass tubular member. Said tubular member may comprise a bung. The bung may be located near a proximal end of the tubular member. A neck portion may define a distal port. Said neck portion may comprise a first coding feature or coded feature. Said coded feature of said neck may comprise at least one of or a plurality of, preferably axial, grooves. A ferrule may be configured to cooperate with the first coding feature of the neck portion.

According to an embodiment, a pierceable septum may be positioned over said distal port. Said pierceable septum may comprise a second coding feature. Said second coding feature may be configured to cooperate with said first coding feature of said neck portion. Said coding feature of said septum may comprise, preferably axial, grooves.

According to an embodiment, the ferrule may be configured to cooperate with a coding feature provided by said drug delivery device. In particular, said ferrule may be configured to cooperate with a coding feature provided by a cartridge holder of said drug delivery device.

According to an embodiment, said drug delivery device comprises a pen type drug delivery device.

Another arrangement comprises a cartridge for use with a drug delivery device. The device may deliver at least one drug. The cartridge may comprise a tubular member. The tubular member may comprise a neck portion. The neck portion may define a distal port. A pierceable septum may be positioned over the distal port. A ring, preferably a support ring, may comprise at least one coding feature. A ferrule may be positioned over the support ring and the septum. The ferrule may be configured to cooperate with the at least one coding feature of the support ring.

According to an embodiment, said ferrule comprises a coding feature for cooperating with a cartridge holder of said drug delivery device.

According to an embodiment, said ferrule is geometrically configured to identify a medicament contained within the cartridge. Additionally or alternatively, said ferrule may be geometrically configured so as to cooperate with a corresponding feature provided by a drug delivery device. Said geometrical shape may comprise a beveled edge. The beveled edge may be located near a distal end of a main body of said ferrule.

A further aspect relates to a drug delivery device. The device may comprise the previously described ferrule. The device may comprise a cartridge. The ferrule may be, releasably or permanently, connected to the cartridge. The device may comprise a corresponding coding feature. The corresponding coding feature may cooperate with the coding feature provided on the ferrule.

According to an embodiment, the device may comprise at least one of or both of a dose setting member and a cartridge holder. The cartridge may be arranged within the cartridge holder. The corresponding coding feature may be provided by at least one of the cartridge holder and the dose setting member.

According to a preferred embodiment, a ferrule is provided which is configured for use with a cartridge for a drug delivery device. Said ferrule comprises a main body defining a bore, said main body extending from a proximal end to a distal end, said bore being configured for receiving the cartridge. Said ferrule comprise at least one coding feature provided along at least a portion of the main body. The at least one coding feature is configured to cooperate with a corresponding coding feature provided by the drug delivery device.

According to a preferred embodiment, a ferrule is provided for use with a cartridge, said ferrule comprising a main body defining a bore, said main body extending from a proximal end to a distal end, said bore configured for receiving said cartridge. The ferrule further comprises a side wall extending from said proximal end and said distal end, a pass through defined by said distal end, and a coding feature provided along at least a portion of said main body of said ferrule. Said coding feature cooperates with a corresponding coding feature provided by a drug delivery device.

According to a preferred embodiment, a ferrule for use with a cartridge is provided, said ferrule comprising a main body defining a bore, said main body extending from a proximal end to a distal end, said bore configured for receiving said cartridge. The ferrule comprises a proximal flange located near said proximal end of said main body. The ferrule comprises a side wall extending from said flange towards said distal end. The ferrule comprises a pass through defined by said distal end. The ferrule comprises a coding feature provided along said distal end of said main body of said ferrule. Said coding feature cooperates with a corresponding coding feature provided by a drug delivery device.

According to a preferred embodiment, a cartridge for use with a drug delivery device is provided, the cartridge being configured for delivering at least one drug. The cartridge comprises a neck portion, the neck portion comprising at least one first coding feature. The cartridge comprises the previously described ferrule. The ferrule is configured to mechanically cooperate with the first coding feature of the neck portion.

According to a preferred embodiment, a cartridge for use with a drug delivery device is provided, said cartridge comprising a tubular barrel, said tubular barrel comprising a bung located near a proximal end of said tubular barrel. The cartridge comprises a neck portion defining a distal port. Said neck portion comprises a first coding feature. The cartridge comprises a ferrule configured to cooperate with said first coding feature of said neck portion.

According to a preferred embodiment a cartridge for use with a device that delivers at least one drug is provided. Said cartridge comprises a cartridge, said cartridge comprising a neck portion defining a distal port. Said cartridge comprises a pierceable septum positioned over said distal port. Said cartridge comprises a ring comprising at least one coding feature. Said cartridge comprises a ferrule positioned over said ring and said septum.

According to a preferred embodiment, a cartridge for use with a device that delivers at least one drug is provided, said cartridge comprising a cartridge comprising a neck portion defining a distal port. Said cartridge comprises a pierceable septum positioned over said distal port. Said cartridge comprises a ferrule positioned over said septum. Said ferrule is geometrically configured to identify a medicament contained within the cartridge.

According to a preferred embodiment, a drug delivery device is provided which comprising the previously described ferrule and a cartridge. The ferrule is connected to the cartridge. The device comprises a corresponding coding feature cooperating with the coding feature provided on the ferrule.

These as well as other advantages of various aspects of the present disclosure will become apparent to those of ordinary skill in the art by reading the following detailed description, with appropriate reference to the accompanying figures.

### BRIEF DESCRIPTION OF THE FIGURES

Exemplary embodiments are described herein with reference to the figures, in which:

FIG. 1 illustrates an exemplary pen-type drug delivery device;

FIG. 2A illustrates a cartridge that may be loaded into a cartridge holder of the pen-type drug delivery device illustrated in FIG. 1;

FIG. 2B illustrates a perspective view of a distal end of the glass cartridge illustrated in FIG. 1 without the ferrule and without the pierceable membrane;

FIG. 3 illustrates a first arrangement of a coded ferrule for use with a cartridge that may be used with a pen-type drug delivery device, such as the drug delivery device illustrated in FIG. 1;

FIG. 4 illustrates the ferrule illustrated in FIG. 3 mounted on a cartridge and partially inserted into a cartridge holder;

FIG. 5 illustrates a second arrangement of a coded ferrule for use with a cartridge that may be used with a pen-type drug delivery device, such as the drug delivery device illustrated in FIG. 1;

FIG. 6 illustrates the ferrule illustrated in FIG. 5 mounted on a cartridge and partially inserted into a cartridge holder;

FIG. 7 illustrates a second arrangement of a coded ferrule for use with a cartridge that may be used with a pen-type drug delivery device, such as the drug delivery device illustrated in FIG. 1;

FIG. 8A illustrates yet another arrangement of a coded ferrule for use with a cartridge that may be used with a pen-type drug delivery device, such as the drug delivery device illustrated in FIG. 1;

FIG. 8B provides a cross-sectional illustration of the ferrule arrangement illustrated in FIG. 8A;

FIG. 9 illustrates a yet another arrangement of a coded ferrule for use with a cartridge that may be used with a pen-type drug delivery device, such as the drug delivery device illustrated in FIG. 1;

FIG. 10 illustrates the ferrule illustrated in FIG. 9 mounted on a cartridge;

FIG. 11 illustrates the ferrule illustrated in FIG. 9 mounted on a cartridge and partially inserted into a cartridge holder;

FIG. 12 illustrates a yet another arrangement of a coded ferrule for use with a cartridge that may be used with a pen-type drug delivery device, such as the drug delivery device illustrated in FIG. 1;

FIG. 13 illustrates the coded ferrule of FIG. 12 mounted on a cartridge;

FIG. 14 illustrates the ferrule illustrated in FIG. 13 mounted on a cartridge and partially inserted into a cartridge holder;

FIG. 15 illustrates a yet another arrangement of a coded ferrule for use with a cartridge that may be used with a pen-type drug delivery device, such as the drug delivery device illustrated in FIG. 1;

FIG. 16 illustrates the ferrule illustrated in FIG. 15 mounted on a cartridge;

FIG. 17 illustrates a perspective view of the ferrule illustrated in FIG. 16;

FIG. 18 illustrates another arrangement of a coded ferrule for use with a cartridge that may be used with a pen-type drug delivery device, such as the drug delivery device illustrated in FIG. 1;

FIG. 19 illustrates another arrangement of a coded ferrule for use with a cartridge that may be used with a pen-type drug delivery device, such as the drug delivery device illustrated in FIG. 1;

FIG. 20 illustrates a top perspective view of the coded ferrule illustrated in FIG. 19;

FIG. 21 illustrates an alternative reservoir that may be used with another coded ferrule arrangement;

FIG. 22 illustrates yet another coded ferrule arrangement; and

FIG. 23 illustrates an alternative reservoir that may be used with another coded ferrule arrangement.

### DETAILED DESCRIPTION

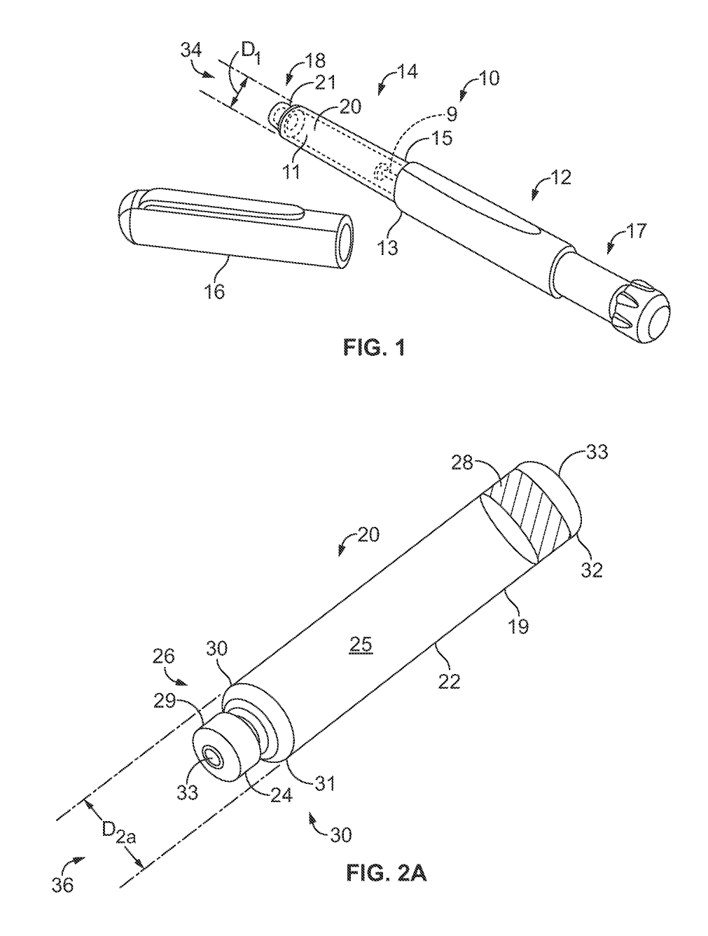

Referring to FIG. 1, there is shown a drug delivery device 10 in the form of a pen-type syringe. This drug delivery device 10 comprises a dose setting mechanism 12, a cartridge holder 14, and a removable cap 16. A proximal end 15 of the cartridge holder 14 and a distal end 13 of the dose setting mechanism 12 may be removably secured together. The pen-type syringe may comprise a re-usable or a disposable pen-type syringe. Where the syringe comprises a re-usable device, the cartridge holder 104 and the dose setting mechanism 102 are removably coupled together. In a disposable device, they are permanently coupled together. In FIG. 1, the dose setting mechanism 12 comprises a piston rod 9, such as a threaded piston rod that rotates when a dose is injected.

To inject a previously set dose, a double ended needle assembly is attached to a distal end 18 of the cartridge holder 14. Preferably, the distal end 18 of the holder 14 comprises a thread 21 (or other suitable connecting mechanism such as a snap lock, snap fit, form fit, or bayonet lock mechanism) so that the needle assembly may be removably attached to the distal end 18 of the holder 14. When the drug delivery device 10 is not in use, the removable cap 16 can be releasably retained over the cartridge holder 14.

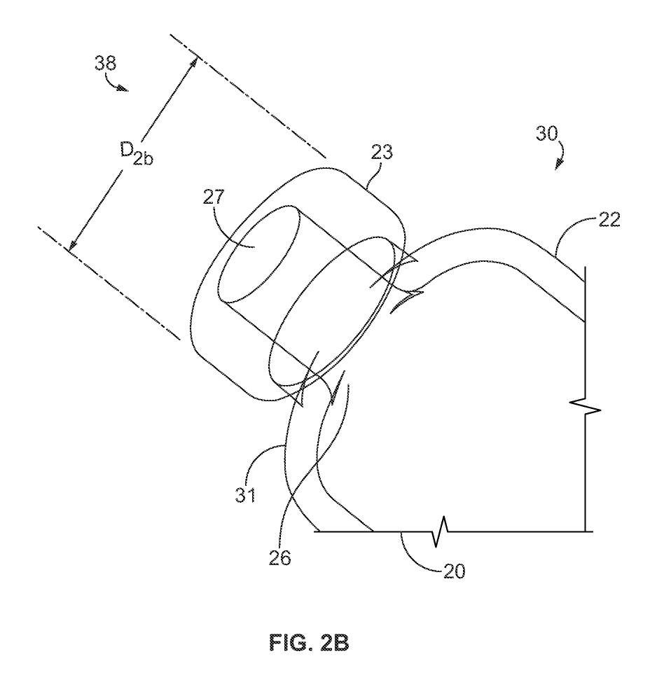

An inner cartridge cavity 11 defined by the cartridge holder 14 is dimensioned and configured to securely receive and retain the cartridge assembly 20 comprising a glass cartridge 20. FIG. 2A illustrates a perspective view of the cartridge assembly 20 that may be used with the drug delivery device 10 illustrated in FIG. 1. FIG. 2B illustrates a perspective view of a distal end 30 of this glass cartridge 20 with the membrane and ferrule removed. Typically, the cartridge 20 is manufactured of glass and includes a generally tubular barrel 22 extending from a distal end 30 to a proximal end 32.

Referring now to FIGS. 2A and 2B, at the distal end 30, the cartridge 20 includes a smaller diameter neck 26 and this neck 26 projects distally from the shoulder 31 of the barrel 22. Preferably, this smaller diameter neck 26 is provided with a large diameter annular bead 23 (shown in FIG. 2B) and this bead 23 extends circumferentially thereabout at the extreme distal end of the neck 26 and defines an opening 27. A pierceable seal, membrane or septum 33 is securely held across the opening 27 by a metallic sleeve or ferrule 24. This ferrule 24, having a smooth outer surface or sidewall 29, may be crimped around the circumferential bead at the distal end of the neck 26 so as to hold the pierceable membrane 33 in place.

The medicament 25 is pre-filled into the cartridge 20 and is retained within this cartridge 20, in part, by the pierceable seal 33, the ferrule 24, and the stopper 28. The stopper 28 is in sliding fluid-tight engagement with the inner tubular wall of the barrel 22. Axially directed forces acting upon the stopper 28 during dose injection or dose administration urges the medication 25 from the cartridge 20 though the double ended needle mounted onto the distal end 30 of the cartridge holder 14 and into the injection site. Such axially forces may be provided by the piston rod 9 working in unison with the dose setting member 12.

A portion of the cartridge holder 14 defining the cartridge holder cavity 11 is of substantially uniform diameter represented in FIG. 1 by D₁ 34. This diameter D₁ 34 is preferably slightly greater than the diameter D₂ 36 of the cartridge 20. The interior of the cartridge holder 14 includes an inwardly-extending annular portion or stop that is dimensioned to prevent the cartridge 20 from moving within the cartridge holder 14. In this manner, when the cartridge 20 is loaded into the cavity 11 of the cartridge holder 14 and the cartridge holder 14 is then connected to the dose setting member 12, the cartridge assembly 20 will be securely held within the cartridge holder cavity 11.

A number of doses of the medicament 25 may be dispensed from the cartridge 20. Preferably, the cartridge 20 contains a type of medicament 25 that must be administered often, such as one or more times a day. One such medicament may be insulin.

The dose setting mechanism 12 comprises a dose setter 17 at the proximal end of the dose setting mechanism 12. In one preferred arrangement, the dose setter 17 is rotated to set a dose. To administer this set dose, the user attaches the needle assembly comprising a double ended needle on the distal end 18 of the cartridge holder 14. In this manner, the needle assembly pierces the seal 33 of the cartridge 20 and is, therefore, in liquid communication with the medicament 25. The user pushes on the dose setter 17 to inject the set dose. The same dose setting and dose administration procedure is followed until the medicament 25 in the cartridge 20 is expended and then a new cartridge must be loaded in the device 10. To exchange an empty cartridge 20, the user is called upon to remove the cartridge holder 14 from the dose setting mechanism 12.

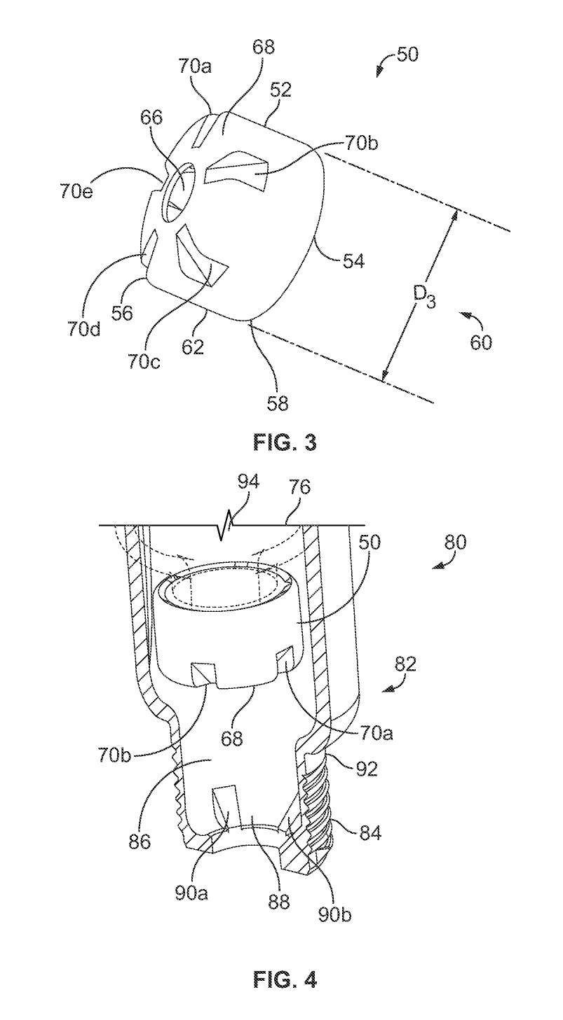

FIG. 3 illustrates a first arrangement of a coded ferrule 50. Such ferrule 50 can be combined with a tubular member (preferably a glass tubular member) and pierceable seal to form a cartridge that may be used with a drug delivery device, such as the pen-type drug delivery device 10 illustrated in FIG. 1. In this arrangement, the coded ferrule 50 comprises a molded coded ferrule rather than a metallic ferrule. Such a molded ferrule may offer a number of advantages. For example, a molded ferrule may enable a more robust and finer coding scheme than it could be applied to a metal ferrule. For example, the ferrule 50 may comprise a plastic such as PP, acetal, polyamide, or, alternatively, a metal such as zinc or magnesium. In addition, the proposed molded ferrule 50 may be used with standard glass cartridges, such as the glass cartridge 20 illustrated in FIGS. 2A and 2B. Because this standard cartridge 20 needs not to be modified, the molded ferrule of the present disclosure may also reduce cost and technical risk. For example, other ferrule coding arrangements described herein may necessitate modification to the glass, which could need development and investment in new cartridge manufacturing production equipment and development of new manufacturing processes.

The ferrule 50 shown in FIG. 3 comprises a cylindrically shaped main body 52 defining a centrally located bore 54. This bore 54 is geometrically configured for receiving a distal end of a standard glass cartridge, such as the distal end of the cartridge 20 illustrated in FIG. 2B. This bore 54 extends from a proximal end 58 to a distal end 56 of the main body 52 and, when a cartridge such as the cartridge 20 illustrated in FIG. 2 is assembled, the bore 54 is positioned over the opening defined by the neck 26 of the cartridge 20. Preferably, the main body 52 has a diameter D₃ 60 that is slightly larger than the diameter of the neck 26 of the cartridge illustrated as D_{2B}. This main body 52 is also dimensioned to fit within an inner cavity of a cartridge holder, such as the inner cavity 11 of the cartridge holder 14 illustrated in FIG. 1. The ferrule 50 further comprises a axially extending wall 62 that extends from the proximal end 58 of the main body 52 towards the distal end 56 of the main body 52.

Near its distal end 56, the ferrule 50 is provided with a pass through 66. In one arrangement, this pass through 66 is sized or configured so that, when the ferrule 50 is positioned over the neck 26 of the cartridge 20, the pass through 66 will expose a portion of a pierceable seal 33 so as to provide a needle assembly access to at least a portion of this seal 33.

Preferably, the molded ferrule 50 comprises coding features and these coding features could be applied in various positions and/or locations on the ferrule. As just one example, in FIGS. 3 and 4, this molded ferrule 50 comprises a coding feature in the form of a plurality of indentations 70a-e provided along an end face 68 of the main body 52. These indentations 70a-e are arranged so that they will cooperate with a cartridge holder that includes a cooperating coding mechanism. Although only five indentations are illustrated in this preferred arrangement, those of skill in the art will recognize alternative indentation arrangements may also be provided, and they may not be equally sized or equally spaced around the circumference.

The ferrule 50 is intended for use with a cartridge holder similar to the cartridge holder 14 of FIG. 1 but a preferred cartridge holder for use with the ferrule 50 would have a slightly modified inner cavity. For example, FIG. 4 illustrates a cross-sectional view of a distal end 82 of a modified cartridge holder 80 that could be used with the ferrule 50. The proximal end (not shown) of the cartridge holder 80 would include a similar releasable connection mechanism (e.g., thread, snap lock, snap fit, bayonet lock, etc.) as the proximal end 15 of the cartridge holder 14 illustrated in FIG. 1.

In this modified cartridge holder 80, the distal end 18 of the cartridge holder 80 comprises a distal threaded section 84 on an outer surface 92 of the holder 80. In addition, the inner cavity 86 of the holder 80 is configured to comprise an inner stop face 88 that has a cooperating coding scheme in the form of a plurality of raised features or protrusions 90a-b. These raised features 90a-b are geometrically configured so as to align with or to cooperate with the plurality of recesses 70a-e provided on the end face 68 of the ferrule 50. For example, FIG. 4 illustrates the molded ferrule 50 illustrated in FIG. 3 mounted on a glass cartridge 76, such as the cartridge illustrated in FIG. 2B. This cartridge 76 and ferrule combination 50 is illustrated as being partially inserted into the cartridge holder 80 wherein two raised features or protrusions 90a-b are illustrated. These features 90a-b are configured so as to engage two recesses or indentations 70a-b on the end face 68 of the ferrule 50.

In this manner, when the cartridge 76 carrying the ferrule 50 is inserted into the cartridge holder 80, the indentations 70a-b of ferrule 50 may cooperate with the distal end projections 90a-b such that the ferrule 50 and, therefore, the cartridge 76 can reside in a final seated position. In this final seated position, the end face 68 of the ferrule 50 fully abuts the inner stop face 88 of the cartridge holder 80.

The distal end 82 of the cartridge holder 80 is intended for use with a standard double ended needle wherein this needle assembly comprises a hub having an internal thread. As such, an outer surface 92 of the cartridge holder 80 is provided with the outer thread 84 that receives such a hub of the double ended needle. Such an outer thread 84 could comprise a single or a double start outer thread. In addition, when such double ended needle is mounted onto the cartridge holder distal end, the piercing proximal needle projects through the pass through 66 and into a pierceable seal of the cartridge 76. As such, when the cartridge 76 comprising the ferrule 50 is fully inserted into the cartridge holder 80 and the double ended needle assembly is mounted on the distal end 82 of the cartridge holder 80, the piercing needle pierces the membrane so as to be in fluid communication with a medicament 94 contained in the cartridge 76.

One advantage of utilizing a plurality of indentations 70a-e along an end face of the ferrule 50 in combination with the raised features 90a-e is that such a coding scheme may pevent a standard cartridge assembly (such as that illustrated in FIG. 2A) from being used with the cartridge holder 80 illustrated in FIG. 4. For example, if a user tried to insert the cartridge 20 of FIG. 2A into the cartridge holder 80 of FIG. 4, the protrusions 90a-b would prevent the cartridge 20 from residing in a final seated position. Consequently, since the end face of the ferrule 24 of the cartridge 20 would not fully abut the inner stop face 88 of the cartridge holder 80, fluid communication between an attached needle assembly and the medicament 25 contained within the cartridge 20 would be prevented. In addition, with an incorrect cartridge, the proximal end of the incorrect cartridge would protrude too far out of the holder 80, thereby preventing assembly of the cartridge holder 80 to the dose setting member 12 of the device 10.

Another advantage of the described indentation/protrusion coding scheme is that it may prevent rotation of the cartridge 76 when the double ended needle is mounted onto the distal end of the cartridge holder 80 which may occur by threading the needle assembly onto the receiving thread 84 at the distal end of the cartridge holder 80. In addition, it may also prevent rotation of the coding relative to cartridge 76. While inserting the cartridge 76 into the holder 80, the user is required to hold the cartridge 76 in order to align the coding features. Therefore, if the coding features were allowed to rotate relative to cartridge 76, it would be difficult to align these coding features.

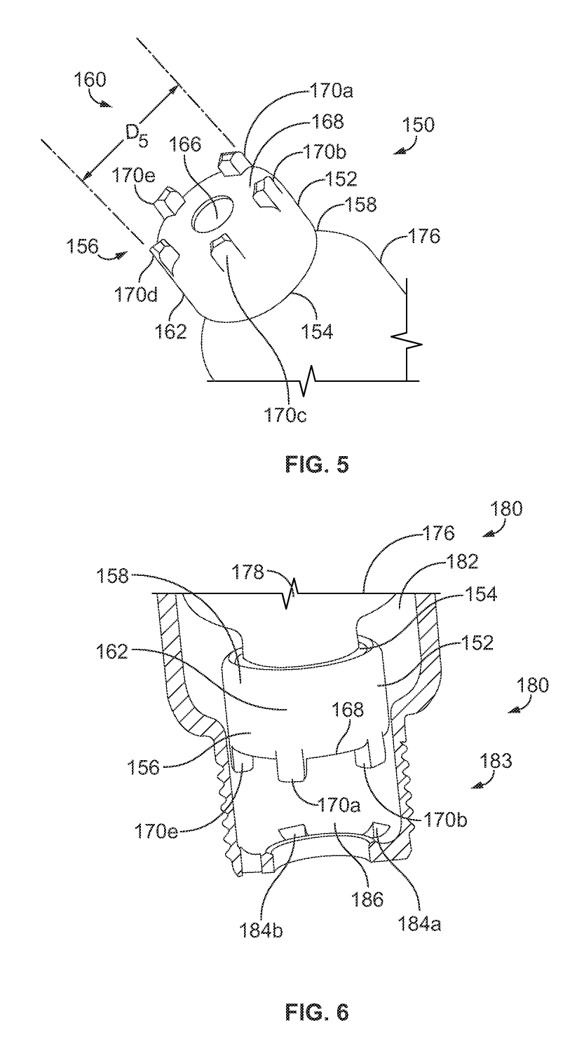

FIG. 5 illustrates a second arrangement of a coded ferrule 150 for use with a cartridge that may be used with a pen-type drug delivery device, such as the drug delivery device 10 illustrated in FIG. 1. Similar to the ferrule 50 illustrated in FIGS. 3 and 4, this ferrule configuration 150 comprises a cylindrically shaped main body 152 defining a centrally located bore 154. This bore 154 is geometrically configured for receiving a distal end of a standard cartridge 176 containing a medicament 178, such as the distal end of the cartridge 20 illustrated in FIG. 2B. This bore 154 extends from a proximal end 158 to a distal end 156 of the main body 152. When the cartridge is inserted, the aperture is positioned over the opening defined by the neck of the cartridge 176. Preferably, this main body 152 has a diameter D₅ 160 that is slightly larger than the diameter of the neck of the cartridge, such as D_{2B} 38 as illustrated in FIG. 2B. As illustrated in FIG. 6, the main body 152 is dimensioned to fit within an inner cavity 182 of a cartridge holder 180, such as the holder illustrated in FIG. 1. The ferrule 150 further comprises an essentially smooth axially extending wall 162. Wall 162 extends from the proximal end of the main body 152 towards the distal end 156 of the main body 152. Near its distal end 156, the ferrule 150 is provided with a pass through 166.

Ferrule 150 further comprises a coding feature in the form of a plurality of protrusions 170a-e provided along an end face 168 of the main body 152. These protrusions 170a-e are arranged so that they will cooperate with a cartridge holder 180 that includes a cooperating coding mechanism. For example, ferrule 150 is intended for use with a cartridge holder 180 similar to the cartridge holder 14 of FIG. 1 but with a modified inner cavity 182. FIG. 6 illustrates the ferrule 150 illustrated in FIG. 5 mounted on a cartridge 176 and partially inserted into such a modified cartridge holder 180.

As can be seen from FIG. 6, the cartridge holder 180 comprises a plurality of recessed features 184a-b located within the cartridge holder inner cavity 182 and located near the distal end 183 of the holder stop face 186. In this manner, when the cartridge 176 carrying the ferrule 150 is inserted into the cartridge holder 180, the projections of ferrule 150 cooperate with the distal end recessed features 184a-b. The ferrule 150 and, therefore, the cartridge 176 can reside in a final seated position where the ferrule end face 168 fully abuts the inner stop face 186 of the cartridge holder 180.

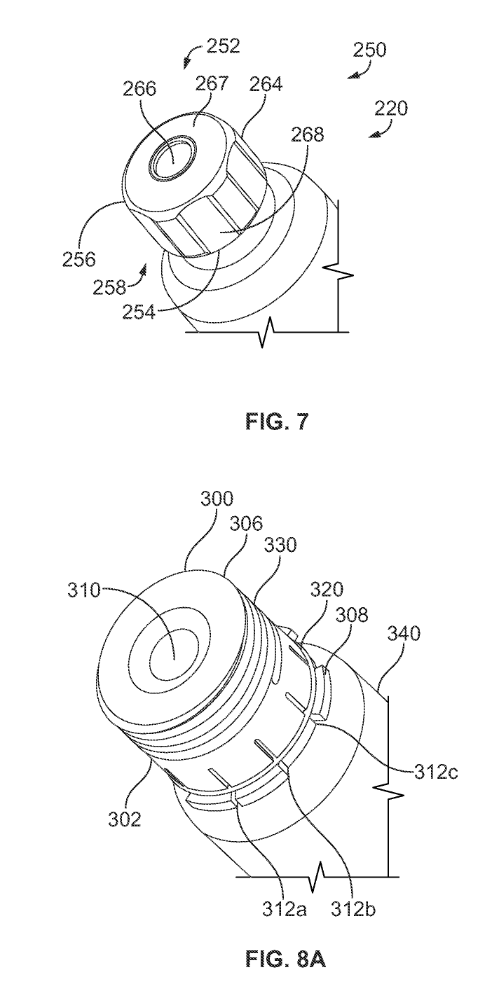

Alternatively, coding features could be provided along a sidewall of a ferrule. For example, FIG. 7 illustrates another arrangement of a coded ferrule 250 for use with a cartridge that may be used with a pen-type drug delivery device, such as the drug delivery device 10 illustrated in FIG. 1.

In this alternative arrangement, similar to the ferrule arrangements 50 and 150, ferrule 250 comprises a main body 252 defining a centrally located bore 254 configured for receiving a distal end of a standard cartridge. This bore 254 extends from a proximal end 258 to a distal end 256 of the main body 252 and, when a cartridge such as the cartridge 20 illustrated in FIG. 2 is assembled utilizing ferrule 250, the aperture is positioned over the opening defined by the neck 26 of the cartridge 20. Preferably, this ferrule main body 252 has a geometrical configuration that is dimensioned to fit within an inner cavity of a cartridge holder. Near its distal end 256, the ferrule 250 is provided with a pass through 266 on its end face 267.

Molded ferrule 250 no longer comprises a smooth side wall but now comprises a coding feature in the form of a side wall 264 having a plurality of axial protrusions and/or indentations 268. Alternatively, the ferrule 250 could be coded by its cross-sectional shape in a transverse plane. These surfaces may be arranged so that they cooperate with a cartridge holder that includes a cooperating coding mechanism. In such a cooperating configuration, the cartridge holder would comprise a plurality of cooperating features located within an inner cavity and located near the distal end of the holder. In this manner, when the cartridge 220 carrying the ferrule 250 is inserted into the cartridge holder, the projections of ferrule 250 cooperate with the distal end recessed features such that the ferrule 250, and, therefore, cartridge assembly 220, can reside in a final seated position with the end face 267 fully abutting the cartridge holder inner stop face.

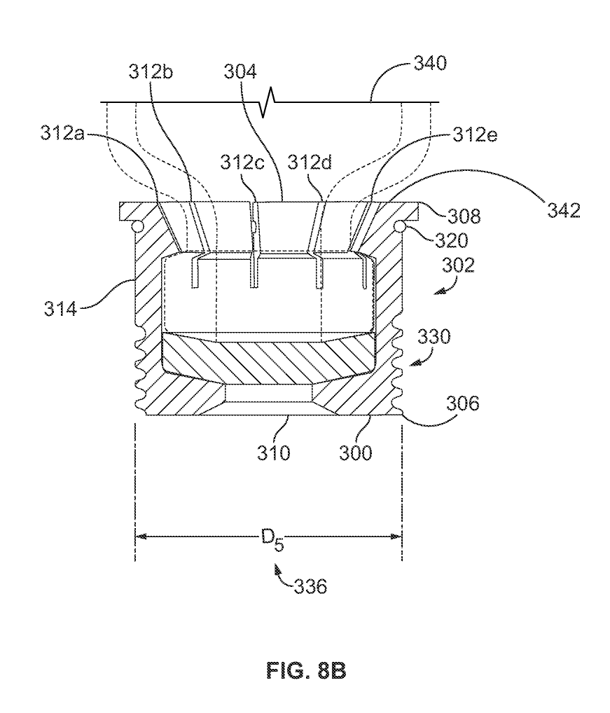

Alternatively, the coding features may be provided along a flange or shoulder of the ferrule, e.g. the shoulder at the proximal end of the ferrule. For example, FIG. 8A illustrates an alternative ferrule arrangement 300 where the coding configurations are provided on a flange 342 of the ferrule 300. FIG. 8B provides cross-sectional illustration of this ferrule arrangement 300. As illustrated, in this arrangement, the ferrule 300 comprises a main body 302 defining a centrally located bore 304. Bore 304 is geometrically configured for receiving a distal end of a cartridge 340. This bore 304 extends from a proximal end 308 to a distal end 306 of the main body 302 and, when a cartridge such as the cartridge illustrated in FIG. 2 is assembled utilizing ferrule 350, the aperture is positioned over the opening defined by the neck of the cartridge. Preferably, this ferrule main body 302 has a geometrical configuration dimensioned to fit within an inner cavity of a cartridge holder. Near its distal end 306, the coded ferrule 300 is provided with a pass through 310.

In one arrangement, the cartridge 340 formed with ferrule 300 is provided with a locking ring 320. This locking ring 320 may be added to the ferrule 300 after the ferrule 300 has been fitted to the cartridge 340. The locking ring 320 may be rectangular in its cross-section and have certain form fit or snap fit features that allow it to be retained on the ferrule 300. One of the advantages of such a locking ring arrangement 320 is that it may be used to increase the rigidity of the ferrule 300 and to increase its retention strength. As illustrated, this locking ring 320 may be provided near the proximal end of the ferrule 300. In addition, the proximal end of the ferrule 300 may be provided with a plurality of slits or cuts 312a-e so as to increase the flexibility of the ferrule 300, thereby making it easier to fit the ferrule 300 over the cartridge neck.

In addition, the ferrule 300 may be provided with a threaded distal end 330. For example, in contrast to the cartridge holder 80 illustrated in FIG. 4 which provides one such threaded section, the ferrule 300 may be provided with a threaded portion 330 along its distal end 306. In this manner, where the ferrule 300 is used with a cartridge holder, a distal end of the cartridge holder will be provided with a pass through so as to allow the threaded section 330 of the ferrule 300 to be accessible to the user so that the user can mount a conventional double ended needle assembly.

In addition, in alternative ferrule arrangements, a chamfer or a ramped/helical surface may be provided on the ferrule, such as along an outer surface 314 of the main body 302 of ferrule 300. One advantage of such a configuration is that it would help a user to align the coding features on the ferrule 300 with the coding features provided in or on the cartridge holder and/or drug delivery device. As just one example, the ferrule 300 may comprise one or more radially outward directed pins. Such pins may have an extent larger than the outer diameter of the cartridge so as to contact a sloped surface along an inner surface of a corresponding cartridge holder. While the cartridge with such a ferrule travels axially into the holder during cartridge loading, the contact between the ferrule and cartridge holder causes the cartridge to rotate, aligning the coding features of the ferrule with the cooperating features provided on the holder.

In the molded ferrule arrangements discussed above, the ferrule may comprise inwardly projecting members located within an inner space of the main body bore. In this manner, the ferrule could be attached to the glass cartridge using these inwardly projecting members so that the inwardly projecting members grip behind the cartridge neck. Alternative attachment methods may also be used.

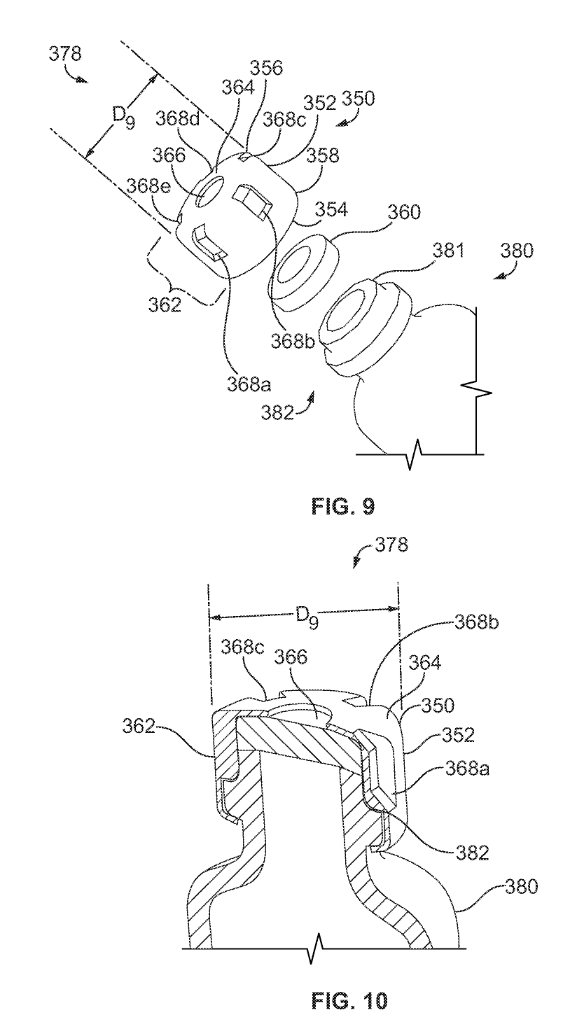

FIG. 9 illustrates a yet another arrangement of a coded ferrule 350 for use with a cartridge 380 that may be used with a drug delivery device, such as the drug delivery device 10 illustrated in FIG. 1. FIG. 10 illustrates the ferrule 350 illustrated in FIG. 9 mounted on the cartridge 380 and FIG. 11 illustrates the ferrule 350 mounted on a cartridge and partially inserted into a cartridge holder 390.

In this arrangement, the coding arrangement is used with cartridge 380 comprising a ferrule 350, a pierceable membrane 360, and a tubular member having a modified neck portion 382. In this arrangement, the ferrule 350 may be metallic or may comprise a mouldable or similar other material as previously described.

The ferrule 350 has a similar construction to ferrule 50 illustrated in FIG. 3. That is, ferrule 350 comprises a cylindrically shaped main body 352 defining a centrally located bore 354 that extends from a proximal end 358 to a distal end 356 of the main body 352. When a cartridge assembly is assembled with such a coded ferrule, the aperture is positioned over the opening defined by the neck of the cartridge. The ferrule 350 further comprises an axially extending wall 362 that extends from the proximal end of the main body 352 towards the distal end 356 of the main body 352. Near its distal end 356, the ferrule 350 is provided with a pass through 366.

One difference between the cartridge 20 that can be used with the ferrule 50 (FIG. 2B and 3) and cartridge 380 for use with the ferrule 350 is that the distal neck portion 382 of the cartridge 380 has been modified. Preferably, in the arrangement of FIG. 9, the distal end 382 of the cartridge 380 is modified to comprise a smaller flange diameter at the distal end 382 of the cartridge 380. By reducing this cartridge flange diameter, this also reduces the overall outer diameter of the ferrule 350. FIG. 9 illustrates the ferrule 350 with a reduced diameter D₉ 378. Moreover, in the example shown, the diameter of the pierceable septum has also been reduced so as to accommodate indentations in the ferrule, but it may have indentations to match those of the ferrule. Alternatively, its outer diameter may be larger than that of the flange 381. In such a configuration, it would be deformed by the coding indentations, and may be more securely retained.

As illustrated in FIG. 10, reducing the cartridge flange diameter enables coding features in the form of indentations 368a-e to be formed near an end face 364 of the ferrule 350. This can be accomplished while maintaining the same diameter for crimping a metallic ferrule or molding such a molded ferrule on the cartridge 380.

By removing a certain amount of cartridge material (e.g., glass, plastic or the like) from the neck portion 382 of the cartridge 380, a user will be prevented from trying to load a larger standard cartridge (such as cartridge 20 in FIG. 2B) into a modified cartridge holder that has been mechanically coded for the smaller diameter of ferrule 350 since the cartridge holder will now have an inner cavity with a smaller inner diameter. Consequently, inadvertent cross use of cartridge assemblies can be reduced.

The ferrule 350 is intended for use with a cartridge holder similar to the cartridge holder 14 of FIG. 1 but with a slightly modified inner cavity. For example, FIG. 11 illustrates a cross-sectional view of a distal end 392 of a modified cartridge holder 390 that could be used with the ferrule 350. The proximal end (not shown) of the cartridge holder 390 would include a similar releasable connection mechanism (e.g., thread, snap lock, snap fit, bayonet lock, etc.).

In this modified cartridge holder 390, the distal end 392 comprises a distal threaded section 394 on an outer surface 396 of the holder. In addition, the inner cavity 398 of the holder 390 is configured to comprise an inner stop face 386 that has a cooperating coding scheme in the form of a plurality of raised features or protrusions 388a-b. These raised features 388a-b are geometrically configured so as to align with or cooperate with the plurality of recesses 368a-e provided on the end face of the ferrule 350. In FIG. 11, this cartridge and ferrule combination is illustrated as being partially inserted into the cartridge holder 390 wherein two raised features or protrusions 388a-b are illustrated and these features 388a-b are configured so as to engage two recesses 368a-b on the ferrule 350.

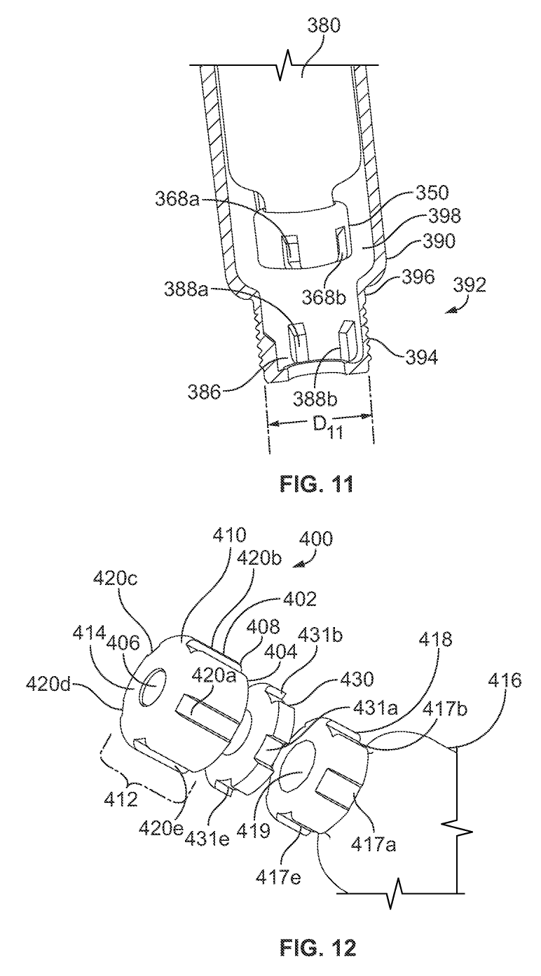

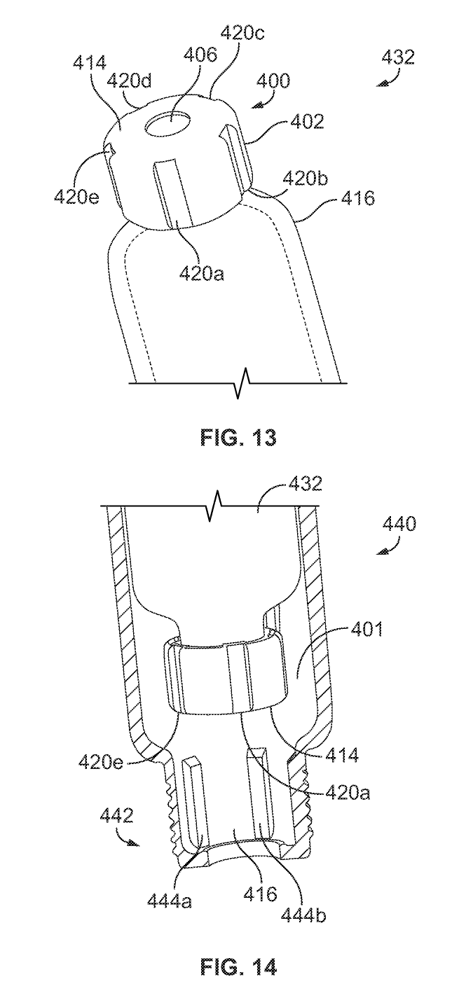

FIG. 12 illustrates a yet another arrangement of a coded ferrule 400 for use with a cartridge that may be used with a pen-type drug delivery device, such as the drug delivery device 10 illustrated in FIG. 1. FIG. 13 illustrates the coded ferrule 400 of FIG. 12 mounted on a coded cartridge 416 and having a coded pierceable membrane 430 between the coded ferrule 400 and the coded cartridge 416.

The ferrule 400 is similar in construction to the coded ferrule illustrated in FIG. 9. As shown in FIG. 12, coded ferrule 400 comprises a cylindrically shaped main body 402 defining a centrally located bore 404. This bore 404 is geometrically configured for receiving a distal end of a standard glass cartridge, such as the distal end of the cartridge 20 illustrated in FIG. 2B. This bore 404 extends from a proximal end 408 to a distal end 410 of the main body 402. When a cartridge such as the cartridge illustrated in FIG. 2 is assembled with this ferrule 400, the aperture is positioned over the opening defined by the neck of the cartridge. This main body 402 is also dimensioned to fit within an inner cavity of cartridge holder, such as the inner cavity 401 of the cartridge holder 440 illustrated in FIG. 14. The ferrule 400 further comprises an axially extending wall 412 that extends from the proximal end of the main body 408 towards the distal end 410 of the main body 402. Near its distal end 410, the ferrule 400 is provided with a pass through 406.

Importantly, the coded ferrule 400 is configured to cooperate with the coded pierceable membrane 430 and the coded cartridge 416. For example, in one arrangement, the coded ferrule 400 comprises a plurality of indentations 420a-e. Similarly, the pierceable membrane 430 may comprise a similar number of indentations 431a-e. As previously described with respect to FIG. 9, the pierceable septum 430 may have the same number of indentations so as to match those of the ferrule 400. Alternatively, the piercable septum 430 may have the same number of indentations as the coded neck 418 of the cartridge 416 but yet fewer indentations than the ferrule 400. In this later configuration, the same cartridge 416 and membrane 430 could be used with a plurality of different coded ferrules 400.

The coded neck 418 of the cartridge 416 also comprises a plurality of indentations 417a-e. As such, when the ferrule 400 is crimpled/molded/fitted over the cartridge 416 to contain the coded membrane 430, these various indentations 417a-e align with one another so as to form a cartridge assembly as illustrated in FIG. 13. One advantage of this coding arrangement is that since this coding feature comprises a plurality of axial grooves provided on the side of the cartridge flange, this configuration removes the need to reduce or alter the flange diameter of the cartridge 416 and septum 430. One advantage of such a coding scheme is that the indentations on cartridge 416 could be standard for a wide range of drugs, but the ferrule 400 may only have some of the indentations, so that the ferrule 400 (and not the cartridge 416 or member) would be coded to a specific drug.

Another advantage of such a coding arrangement is that it allows for longer coding indentations to be used in the cartridge holder 416. For example, FIG. 14 illustrates the assembly 432 illustrated in FIG. 13 partially inserted into a cartridge holder 440. The distal end 442 of the holder 440 comprises a stop face. FIG. 14 illustrates the cartridge assembly 432 partially inserted into the cartridge holder 440 wherein two raised features or protrusions 444a-b are illustrated and these features 444a-b are configured so as to cooperate with two recesses or indentations 420a-b on the end face 414 of the ferrule 400. When the cartridge assembly 432 carrying the ferrule 400 is inserted into the cartridge holder 440, the indentations 420a-b cooperate with the distal end projections 444a-b such that the cartridge 432 can reside in a final seated position with the ferrule end face 414 fully abutting the inner stop face 416 of the cartridge holder 440.

Increasing the length of such coding protrusions 444a-b increases a potential ‘stand-off’ distance if an incorrect cartridge is inserted into the cartridge holder 440. That is, the stand-off distance being the distance between the cartridge and the final seated position where the end face of the ferrule 400 abuts the end stop of the cartridge holder inner cavity.

In the arrangement illustrated in FIGS. 12-14, the ferrule 400 may be metal or a molded polymer. Alternatively, the ferrule may comprise a heat-shrink material. Such a heat shrink material could reduce tolerances thus allowing the ferrule 400 to grip the distal bead of the cartridge 416 more closely. Moreover, where the ferrule 400 comprises a malleable or a metallic material, the ferrule 400 could be manufactured first, the cartridge 416 could be fabricated, and then the coding could be pressed into the ferrule end face after it has been fitted onto the cartridge 416. Alternatively, the coding features could be formed by heat causing material to flow into any coding grooves on the cartridge 416.

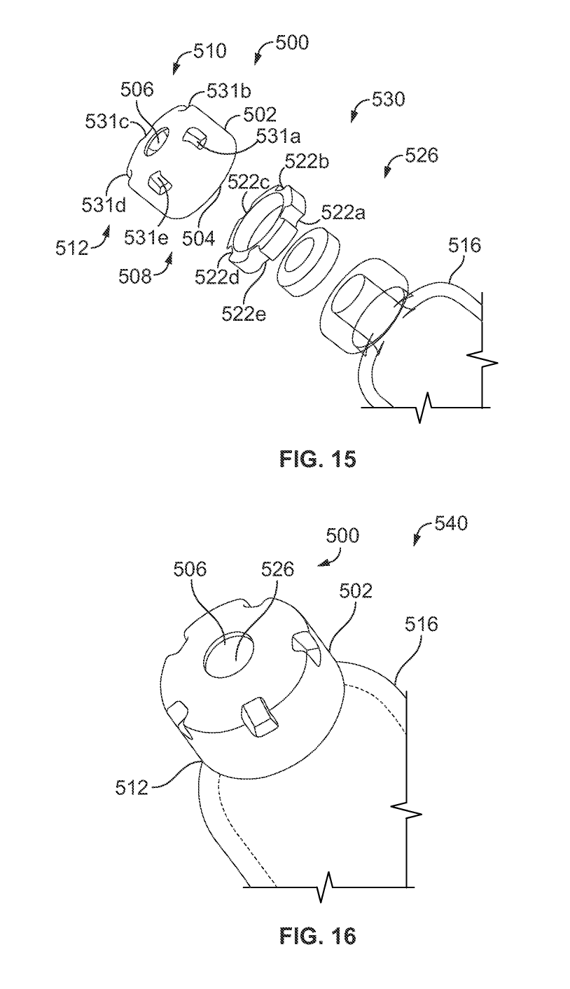

FIG. 15 illustrates a yet another arrangement of a coded ferrule 500 for use with a cartridge that may be used with a pen-type drug delivery device, such as the drug delivery device 10 illustrated in FIG. 1. FIG. 16 illustrates the coded ferrule 500 of FIG. 15 mounted on a cartridge 516 and having a coded support ring 530 between the cartridge 516 and the coded ferrule 500.

The ferrule 500 is similar in construction to the previously described coded ferrules. For example, as shown in FIG. 15, coded ferrule 500 comprises a cylindrically shaped main body 502 defining a centrally located bore 504. This bore 504 is geometrically configured for receiving a distal end of a cartridge, such as the distal end of the cartridge 20 illustrated in FIG. 2B. This bore 504 extends from a proximal end 508 to a distal end 510 of the main body 502. When a cartridge such as the cartridge illustrated in FIG. 2 is assembled, the aperture is positioned over the opening defined by the neck of the cartridge. This main body 502 is dimensioned to fit within an inner cavity of a cartridge holder. The ferrule 500 further comprises an axially extending wall 512 that extends from the proximal end of the main body 502 towards the distal end 510 of the main body 502. Near its distal end 510, the ferrule 500 is provided with a pass through 506.

Importantly, the coded ferrule 500 is configured to cooperate with a coded support ring 530 and the pierceable membrane 526. For example, in one arrangement, the coded ferrule 500 comprises a plurality of indentations 531a-e. Similarly, the coded support ring 530 comprises a plurality of indentations 522a-e. As such, when the ferrule 500 is crimped/molded/fitted over the cartridge 516 to contain the coded support ring 530 and the membrane 526, the indentations of the coded ferrule 500 and the support ring 530 align with one another so as to form a cartridge assembly 540 as illustrated in FIG. 16.

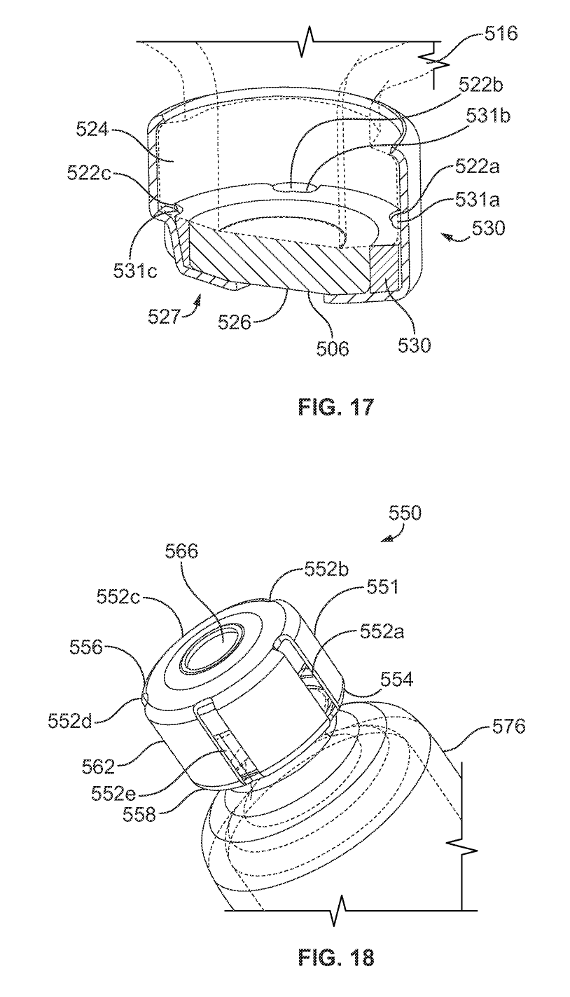

FIG. 17 illustrates a partial perspective view of the coded ferrule 500 and the support ring interface. As shown, the inner indentations 531a-c of the coded ferrule 500 cooperate with the recesses 522a-c provided along the external circumference of the support ring 530. This support ring 530 further defines an inner bore 527 and the pierceable membrane 526 is retained in the recess in part by the ferrule 500 being crimped over a bead 524 of the cartridge 516.

One advantage of such a coded support ring arrangement is that, since the coding features are provided in the form of a plurality of axial grooves provided on the support ring 530, there is no longer a need to reduce the flange diameter of the cartridge 516. Consequently, standard type cartridges may be used with this coding arrangement. Another advantage of this coding configuration is that the addition of the coded support ring 530 increases the robustness of the coded ferrule 500.

In addition, the ferrule coding features may be formed on the ferrule 500 before the ferrule 500 is crimped or fitted over the cartridge neck. Alternatively, the coding features may be formed after the cartridge assembly is assembled. Although the glass cartridge is shown as having a standard neck, the support ring 530 could also sit on a shoulder formed in the cartridge (as illustrated in FIG. 9).

FIG. 18 illustrates another arrangement of a coded ferrule 550 for use with a cartridge that may be used with a pen-type drug delivery device, such as the drug delivery device 10 illustrated in FIG. 1.

Similar to the previously described ferrules, this ferrule configuration 550 comprises a cylindrically shaped main body 551 defining a centrally located bore 554. This bore 554 is configured for receiving a distal end of a cartridge 576 and extends from a proximal end 558 to a distal end 556. When a cartridge is inserted, the aperture is positioned over the opening defined by the neck of the cartridge 576. This main body 551 is dimensioned to fit within an inner cavity of a cartridge holder.

The ferrule 550 further comprises an essentially smooth axially extending wall 562. Wall 562 extends from the proximal end of the main body 551 towards the distal end 556 of the main body 551. Near its distal end 556, the ferrule 550 is provided with a pass through 566. Ferrule 550 further comprises coding features in the form of a plurality slots 552a-e provided in the axially extending wall 562. These coding features 552a-e cooperate with appropriate sized and configured geometrical projections provided within the internal cavity of the cartridge holder. One advantage of such an arrangement is that no change is required to a standard cartridge and, therefore, there would be no additional development and/or investment. There would also be no change to a standard septum so that additional drug compatibility testing would not be needed. In addition, since the outer diameter of the cartridge 576 would remain unchanged, there would be no impact on the overall size of the drug delivery device. Moreover, longer coding features could be used, thereby making it more obvious to a user if he or she attempts to load an incorrect cartridge. In addition, because material has been removed from the ferrule, a small inner cavity of the cartridge holder could be used and, therefore, a cartridge having a standard ferrule would not be properly seated within such a cartridge holder.

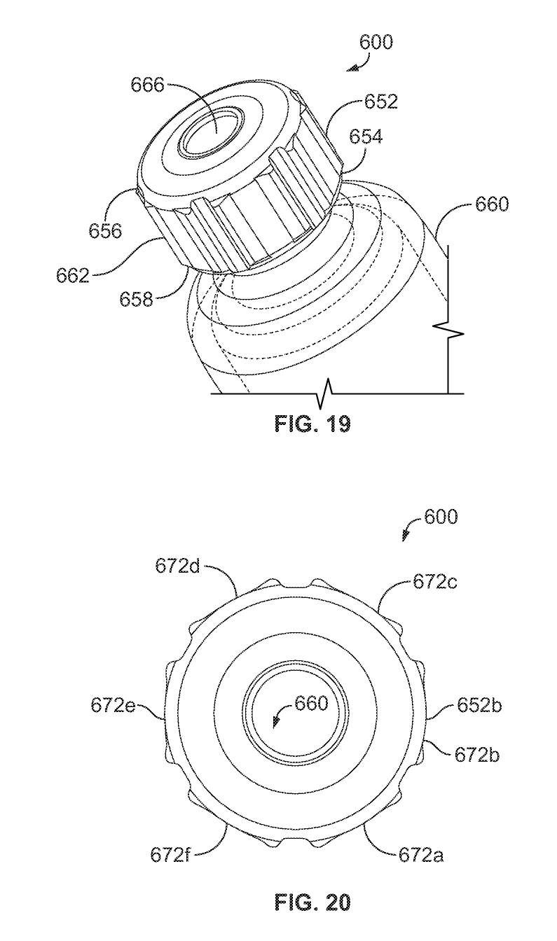

FIG. 19 illustrates yet another arrangement of a coded ferrule 600 for use with a cartridge that may be used with a pen-type drug delivery device, such as the drug delivery device 10 illustrated in FIG. 1. FIG. 20 illustrates a top perspective view of the coded ferrule 600 illustrated in FIG. 19. Similar to the previous ferrule arrangements, ferrule 600 comprises a main body 652 defining a centrally located bore 654. Bore 654 is geometrically configured for receiving a distal end of a standard glass cartridge. This bore 654 extends from a proximal end 658 to a distal end 656 of the main body 652. When a cartridge assembly is assembled utilizing ferrule 600, the aperture is positioned over the opening defined by the neck of the cartridge. Near its distal end 656, the ferrule 600 is provided with a pass through 666.

Preferably, this ferrule main body 652 has a geometrical configuration dimensioned to fit within an inner cavity of a cartridge holder. The ferrule main body 652 further comprises an axially extending wall 662 that extends from the proximal end 658 to the distal end 656 of the main body 652. This axially extending wall 662 comprises coding features in the form of a plurality of thick and thin sections 672a-f, which may be pressed into the ferrule material. These coding features 672a-f cooperate with appropriate sized and configured geometrical recesses provided within the internal cavity of the cartridge holder.

Although aimed primarily at the insulin market, the disclosed coded ferrule may apply to other drugs. The disclosure may apply to various devices, including the following examples:

An injector pen with a cartridge (e.g. 3 ml cylindrical glass cartridge) and a separate holder.

An injector pen with a cartridge (e.g. 3 ml cylindrical glass cartridge) non-removably retained in a holder, so that the holder will be disposed of with the primary pack.

An injector pen where the primary pack attaches directly to the pen, e.g. an injection-molded polymer cartridge.

Any drug delivery device with any type of primary pack, e.g. inhaler, pouch.

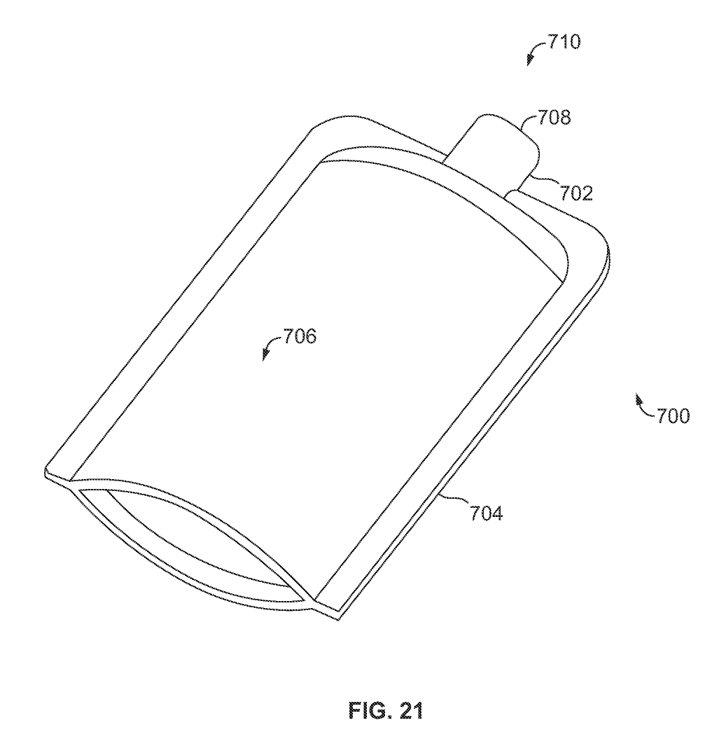

In other situations, the disclosed coding system may apply to any drug delivery device, with any type of reservoir or primary pack, e.g. inhaler, pouch. For example, FIG. 21 illustrates a drug reservoir 700 comprising a vessel 704 that contains a medicament 706. A stopper 708 is provided along a distal end of the vessel 704 and is attached to the vessel 704 so as to prevent the medicament 706 from exiting the vessel 704. A coded ferrule 702 is provided on the vessel 704 near an output port 710 of the vessel 704. This output port 710 has a rigid neck and a coded ferrule 702 is provided along this neck.

The disclosed coding system results in a number of advantages. For example, the proposed coded ferrule arrangements assist a user to distinguish between medicaments, thereby helping to ensure that a drug delivery device can only be used with a medicament for which the device is intended. Therefore, with the system applied to a cartridge, the cartridge is prevented from being confused with any other drug by loading a cartridge with an incorrect or unwanted interface. The disclosed coded ferrule prevents a user from completing one or more of the following actions: fully inserting the cartridge into an incorrect cartridge holder or attaching the cartridge and/or the cartridge holder onto an incorrect dose setting mechanism.

The disclosed coded ferrule also results in a low cost coding mechanism since the proposed ferrules do not require a large number of parts and can be manufactured in a cost effective manner. Moreover, there are quite a large number of different ferrule coding configurations between the ferrule and the cartridge holder that may be used. Consequently, with the proposed coded ferrule schemes, a large number of medicaments can be distinguished from one another. In addition, with the presently disclosed coding schemes, if a user attempts to load an incorrect cartridge into a cartridge holder designed for a different cartridge, the user will be alerted at an early stage of the assembly process.

In addition, The disclosed system can be used to prevent errors during manufacturing, when inserting cartridge into disposable cartridge holders or disposable devices.

Exemplary embodiments of the present disclosure have been described. However, as those of skill in the art will recognize certain changes or modifications to such arrangements may be made. As just one example, certain coding elements of one of the preferred arrangements discussed herein may be taken from one arrangement and combined with certain coding arrangements of other arrangements.

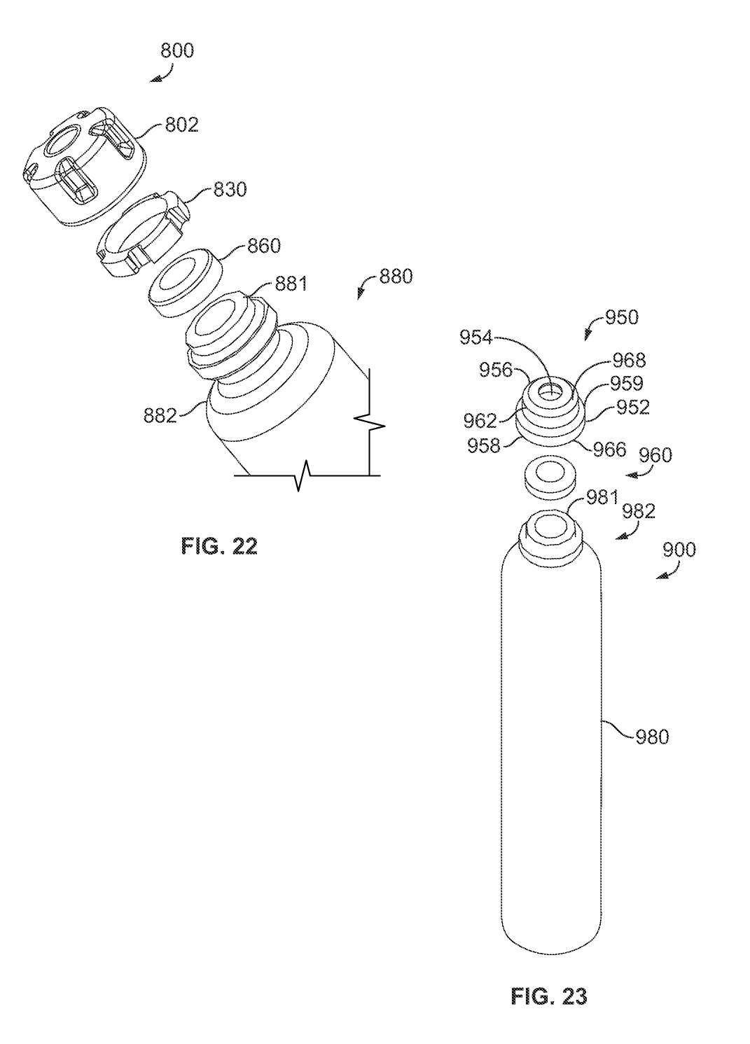

As merely one example, FIG. 22 illustrates one such alternative coding arrangement 800. This coding arrangement 800 represents a combination coding arrangement of the coding scheme illustrated in FIG. 9 combined with that coding scheme illustrated in FIG. 15. In such a combined arrangement, the coding scheme would comprise a coded ferrule 802, a coded permeable member 830, a ring 860, and a cartridge 880. Such cartridge 880 would comprise a neck 882 having a reduced diameter 881.

As yet another example, FIG. 23 illustrates another such alternative arrangement 900. This coding arrangement 900 represents an alternative coding arrangement of the coding scheme illustrated in FIG. 9. In this arrangement, the coding arrangement is used with a cartridge 980 comprising a ferrule 950, a pierceable membrane 960, and a tubular member having a modified neck portion near the distal end 982 of the cartridge 980. As before, the ferrule 950 may be metallic or may comprise a mouldable or similar other material as previously described. In this arrangement, the cartridge 980 for use with the ferrule 950 has been modified to comprise a smaller flange diameter 981 at the distal end 982 of the cartridge 980.

The ferrule 950 has a generally similar construction to ferrule 350 illustrated in FIG. 9. That is, ferrule 950 comprises a cylindrically shaped main body 952 defining a centrally located bore 966 that extends from a proximal end 958 to a distal end 956 of the main body 952. When the cartridge 980 is assembled with such a coded ferrule 950, the aperture is positioned over the opening defined by the neck of the cartridge 980. The ferrule 950 further comprises a proximal flange 959 adjacent an axially extending wall 962. This wall 962 extends from the proximal flange 958 of the main body 952 towards the distal end 956 of the main body 952. Near its distal end 956, the ferrule 950 is provided with a beveled edge 968 and a pass through 954.

Those skilled in the art will understand, however, that further changes, modifications, revisions and/or additions may be made to the presently disclosed arrangements without departing from the true scope and spirit of the present disclosure, which is defined by the claims.

The scope of the disclosure is defined by the content of the claims. The disclosure is not limited to specific embodiments but comprises any combination of elements of different embodiments. Moreover, the disclosure comprises any combination of claims and any combination of features disclosed by the claims.

1-19. (canceled)

20. A cartridge for use with a drug delivery device, the cartridge being configured for delivering at least one drug, the cartridge comprising:
a neck portion, the neck portion comprising at least one first coding feature, and
a ferrule comprising:
a main body defining a bore, said main body extending from a proximal end to a distal end, said bore being configured for receiving the cartridge,
at least one coding feature provided along at least a portion of the main body,
wherein the at least one coding feature is configured to cooperate with a corresponding coding feature provided by the drug delivery device, and
wherein the ferrule is configured to cooperate with the first coding feature of the neck portion of the cartridge.

21. The cartridge according to claim 20, wherein the ferrule comprises a molded ferrule or a metallic ferrule.

22. The cartridge according to claim 20, wherein the main body of the ferrule comprises a side wall, and wherein the coding feature of the ferrule is provided along at least one of or both of the side wall of the main body and an end face of the main body.

23. The cartridge according to claim 20, wherein the coding feature of the ferrule comprises at least one of an indentation and a protrusion.

24. The cartridge according to claim 20, wherein the coding feature of the ferrule comprises at least one slot or at least one vertical wall of the main body, the vertical wall comprising a varying thickness.

25. The cartridge according to claim 24, wherein the vertical wall comprises at least one flat surface.

26. The cartridge according to claim 20, wherein the ferrule comprises a flange, and wherein the coding feature of the ferrule is provided near the flange.

27. The cartridge according to claim 20, wherein the coding feature of the ferrule comprises a beveled edge located near the distal end of the main body.

28. The cartridge according to claim 20, wherein the ferrule further comprises a coupling configured for mounting a needle assembly.

29. The cartridge according to claim 20, wherein the coding feature of the ferrule is adapted and arranged to prevent the cartridge from rotating within a cartridge holder of the device and/or to prevent the ferrule from rotating with respect to the cartridge.

30. The cartridge according to claim 20, wherein the ferrule further comprises a pass through defined by the distal end of the ferrule and a membrane arranged between the pass through and the cartridge.

31. The cartridge according to claim 20, wherein at least one protrusion is provided in the bore of the ferrule, the at least one protrusion being adapted and arranged to mechanically cooperate with a neck of the cartridge when the ferrule is mounted onto the cartridge.

32. The cartridge according to claim 20, wherein the main body of the ferrule comprises a length equivalent to a length of a tubular barrel of the cartridge.

33. The cartridge according to claim 20, wherein the neck portion defines a distal port, and wherein the cartridge further comprises a pierceable septum positioned over the distal port,
wherein the pierceable septum comprises a second coding feature which is configured to mechanically cooperate with the first coding feature of the neck portion.

34. The cartridge according to claim 33, wherein the at least one second coding feature and the at least one first coding feature each comprises a plurality of axial grooves.

35. The cartridge according to claim 33, comprising a ring, the ring comprising at least one coding feature, wherein the ferrule is configured to be positioned over the ring and said septum, and wherein the ferrule is configured to mechanically cooperate with the at least one coding feature of the ring.

36. The cartridge according to claim 20, wherein the ferrule is geometrically configured to identify a medicament contained within the cartridge.

37. A drug delivery device comprising the cartridge according to claim 20, wherein the ferrule is connected to the cartridge, and wherein the device comprises a corresponding coding feature cooperating with the coding feature provided on the ferrule.

38. The drug delivery device according to claim 37, comprising at least one of or both of a dose setting member and a cartridge holder, the cartridge being arranged within the cartridge holder, wherein the corresponding coding feature is provided by at least one of the cartridge holder and the dose setting member.
